(19) United States
(12) Patent Application Publication
(10) Pub. No.: US 2020/0246620 A1
(43) Pub. Date: Aug. 6, 2020

(54) Method And Device Of Treatment Of Abdominal And Pelvic Pain With Neuroaugmentation

(71) Applicant: Bruce H. Levin, Philadelphia, PA (US)

(72) Inventor: Bruce H. Levin, Philadelphia, PA (US)

(21) Appl. No.: 16/778,942

(22) Filed: Jan. 31, 2020

Related U.S. Application Data
(60) Provisional application No. 62/799,687, filed on Jan. 31, 2019.

(51) Int. Cl. A61N 1/36

(52) U.S. Cl. CPC A61N 1/36071; A61N 1/3615; A61N 1/36178; A61N 1/36171

(57) ABSTRACT

A method and device for treating a disorder by multiple points of stimulation.

### RELATED APPLICATIONS

This application claims the benefit of U.S. Provisional Application No. 62/799,687 filed on Jan. 31, 2019 and herein incorporated by reference.

### BACKGROUND OF THE INVENTION

The embodiments of the present invention concern devices and methods for achieving multiple points of therapeutic intervention in one or more applications.

### BRIEF SUMMARY OF THE INVENTION

In one embodiment, the present invention provides a method of treating a disorder of interest, utilizing retrograded approach to place a stimulator contact device or component to a position along the nerve root or branch thereof in a distal neurostructures such that neuro-application inhibits the disorder.

In another embodiment, the present invention provides a method of neuroaugmentation that is applied in the epidural space proximal to the distal neural-structure.

In one embodiment, the present invention provides a method of neuroaugmentation that is applied in the blood vessel.

In one embodiment, the present invention provides a method wherein one or more contact, device or stimulator components are located either immediately proximal, distal, or intraparametally.

In one embodiment, the present invention provides a method of neuroaugmentation in which the position of the contact, frequency, intensity, pulse-width, and/or other parameters, are coordinated, varied changed or held steady along different loci on the catheter

In one embodiment, the present invention provides a method of neuroaugmentation in which the position of the contact, frequency, intensity, pulse-width, and/or other parameters, are changed or held steady to minimize motor stimulation.

In one embodiment, the present invention provides a method of neuroaugmentation wherein contact placement will be optimized to avoid motor afferent or efferent neural involvement or stimulation to minimiz motor stimulation.

In one embodiment, the present invention provides a method of neuroaugmentation wherein a contact is paced at any point on a nerve through a lead.

In one embodiment, the present invention provides a method of neuroaugmentation wherein one or more sub-stimulating units may be permanently or reversible attached to a main stimulator catheter or device, catheter, microchip, or other device, to achieve optimal positioning or allowing for the advancement or placement of smaller diameter neurostimulating devices to optimize placement into one or more neuroforamamina, DRG or related structures, in proximity to a peripheral nerve, nerve fiber, or other neuralstructure.

In one embodiment, the present invention provides a method of neuroaugmentation wherein one or more sub-stimulating units may be individually guided, operated or controlled.

In one embodiment, the present invention provides a method wherein major/main catheter, and/or the smaller diameter catheters may be independently steerable. Each may or may not have its own guide wire, and the micro stimulator or catheter may be composed of materials known to one skilled in the state of the art, or may be comprised a gel, soft polymer, or biologic tissue, substrate or other material to optimize effect and safety and tolerability, especially regarding placements across joints or tissues subject to frequent Flexion, extension, rotation, linear or other translation and the like

In one embodiment, the present invention provides a method wherein a guide wire may operate in different directions, rotations, curvatures or arcs.

In one embodiment, the present invention provides a method of treating a disorder of interest, by applying a catheter or stimulator device, through a neural foramen, stimulator or other device that can be placed in or near a nerve plexus, or component thereof, or into an area adjacent peripheral nerve or branch thereof.

In one embodiment, the present invention provides a method wherein a catheter or stimulator is placed more distally along a nerve root, and/or at the level of in the plexus, and/or at the level of sympathetic structure, and/or at the level of a peripheral nerve or nerves.

In one embodiment, the present invention provides a method that is applied independently or in combination with one or more of the methods, comprising 1) usual epidural spinal cord stimulation, and or 2) with proximal nerve root stimulation, and/or 3) dorsal root ganglion stimulation, and/or 4) distal nerve root stimulation and/or 5) plexus or plexus component and/or 6) peripheral nerve structure and/or 7) a component of a component of the autonomic nervous system and/or 8) either one or both of a component of a parasympathetic or sympathetic nervous system with or without a separate more centrally or more peripherally placed stimulation or topical stimulation device or component thereof.

In one embodiment, the present invention provides a method wherein a stimulator acts on a sensory or autonomic nerve but not a motor nerve.

In one embodiment, the present invention provides a method that is applied with inter-dependent or independent, coordinated or non-coordinated, concurrent or non-concurrent, sequential or parallel, application of stimulation with optimization of parameters including but not limited to frequency, band width, amperage, waveform, power, phase, cycle, timing.

In one embodiment, the present invention provides a method using one or more TENS, EMS units, ultrasound, magnetic, bioelectrical, electrical, infrared, laser, maser, sonic, EMF, pulsed EMF or other modality units.

In one embodiment, the present invention provides a method wherein the methodologies may be used to optimize stimulation, or other therapeutic modalities involving placement in or around vascular structures, organs, viscus, body cavities, glandular, GI, or other neural structures or sympathetic ganglia.

In one embodiment, the present invention provides a method of placing a stimulator contact device or component into a distal neural tructure along a nerve root or nerve root branches. The same stimulator contact device or component can also be applied in the epidural space or along any neural structure or in proximity to a blood vessel to obtain the desired effect.

In one embodiment, the present invention provides a method of treating a disorder of interest, utilizing retrograded approach to place a stimulator contact device or component to a position along the nerve root or branch thereof in proximity distal neural structures such that neuromodulation inhibits the disorder.

In one embodiment, the present invention provides a method wherein neuroaugmentation is applied in the epidural space proximal to the distal neural structure.

In one embodiment, the present invention provides a method wherein one or more contact, device or stimulator component whereby such contact is located either centrally, along the CNS or immediately proximal, distal, or intraforaminally positioned, such that the stimulation may or may not affect stimulation of the dorsal root ganglion.

In one embodiment, the present invention provides a method wherein the foramen is at the level of T12, or L1, or L2, or L3, or L4, or L5, or any sacral level and it can be placed unilaterally or bilaterally to affect inhibition the disorder of interest.

In one embodiment, the present invention provides a method of treating a disorder of interest, by applying a catheter or stimulator device, through a neural foramen, stimulator or other device can be placed in or near a nerve plexus or component thereon or into a peripheral nerve thereon.

In one embodiment, the present invention provides a method wherein a catheter or stimulator device is placed more distally along the nerve root, and/or at the level of in the plexus, and/or at the level of sympathetic structure, and/or at the level of peripheral nerve.

In one embodiment, the present invention provides a method wherein a retrograde or any stimulator consists of, or contains one or more of preformed electrode, diode, lead, circuit, integrated circuit, microcircuit, MEMS, wire, micro wire, array, deployable array, carbon fiber, carbon, carbon monolayer silicone, metal, rare metal, structure, circuit, nanotubule, or other microcircuit, machine, power generator, power source, microrotor, magnet, battery, lead, electrode, sensor, conduit for energy or electricity, electromagnetic array, generator of ultrasound, radiowave, radiation, light, laser, maser, or other energy, engineered cell or cells, biomanufactured organelle, modified or native organ or organ component, resevoir of chemotherapeutic agent, radiotherapeutic agent, sensor or RFID device or related device. of MEMS, nanomachine, chip microchip, electrode, stimulator, sensor.

In one embodiment, the present invention provides a method wherein any stimulator contains artificial or biologic components, may include a collagen or collagen like substance, cartilaginous substance, a neural tissue or type tissue, a smooth or skeletal muscle tissue or type tissue, a connective tissue or type tissue, a cartilaginous tissue or type tissue, vascular tissue or type tissue, an endo or epithelial tissue or type tissue.

In one embodiment, the present invention provides a method wherein a component of a stimulator is native or biologic tissue, cultured tissue, genetically engineered or otherwise altered tissue to enhance or otherwise effect certain physical, chemical, structural, conductive or other properties and/or maintain or optimize homeostatic, metabolic, status or function and durability.

In one embodiment, the present invention provides a method wherein the implantation is percutaneous, peri/trans/intravascular, peri/trans/intracavitary, peri/trans/intraluminal, luminal, dural, peridural, sub dural, intradural, intracranial, arachnoid, subarachnoid, meningeal, sub/intra/periventricular, neural, myofascial, adipose, skeletal or smooth muscle, or cardiac structure or tissue or intratissue.

In one embodiment, the present invention provides a method wherein the implantation is following or concurrent with volume clearing procedure via surgery, balloon plasty, ultrasound, heat, electrical, radiofrequency or other ablative or radiologic or gamma radiation techniques.

In one embodiment, the present invention provides a method wherein one or more devices are embedded, suspended, placed, or physically associated with a substance or material consisting of conductive or non-conductive liquid, fluid, gel, sol, gelsol, malleable solid, foam, putty, or matrix, cell culture, autologous, homologous, cadaveric tissue, tissue culture, or solidifying substance.

In one embodiment, the present invention provides a method that uses a substance or material that is pH, ion, temperature, light, or chemically dependent for state, volume, size, shape, conductivity, or transmissibility.

In one embodiment, the present invention provides a method that uses a substance or material that is artificial or biologic, is hyaluronic acid, Restylane, hyaluronic acid variant, collagen or collagen like substance, cartilaginous substance, is a native or biologic tissue cultured or harvested, is a neural, smooth or skeletal muscle, connective tissue, cartilaginous, vascular, endo or epithelial tissue.

In one embodiment, the present invention provides a method that uses a substance, device or material that is customized according to MRI, CT, X-ray, Ultrasound or other imaging or structure defining modality is utilized. This may include culture, scaffolding or 3d printing or other manufacturing modality.

In one embodiment, the present invention provides a method that uses a substance, device or material that is genetically engineered or otherwise altered to enhance or otherwise effect certain physical, chemical, structural, conductive or other properties and/or maintain or optimize homeostatic, metabolic, status or durability.

In one embodiment, the present invention provides a method wherein a device is directed into place by magnet or magnetism, electric field, electricity, heat, sound, ultra or infrasound, vibration, buoyancy, or light, concentration gradient, or by microbe, or micromachine.

In one embodiment, the present invention provides a method of stimulating neuro or other biological structures, comprising one or more of electrodes or components placed in a fluid or gel like substance or putty like substance; and a tool with a high strength magnet to guide the said one or more electrode or component to the neurologic or other target.

In one embodiment, the present invention provides a method of stimulating neural or other biological structures wherein the electrode or component is solidified.

In one embodiment, the present invention, wherein the electrode or component is in native form.

In one embodiment, the present invention provides a method of stimulating neural or other biological structures wherein the electrode or component is in series.

In one embodiment, the present invention provides a method of stimulating neural or other biological structures wherein the electrode or component is parallel to each other.

In one embodiment, the present invention provides a method of stimulating neural or other biological structures wherein the electrode or component is within a matrix to fix their positions.

In one embodiment, the present invention provides a method of stimulating neural or other biological structures wherein the electrode or component is magnetic.

In one embodiment, the present invention provides a method of stimulating neural or other biological structures wherein the electrode or component is ferrous.

In one embodiment, the present invention provides a method of stimulating neural or other biological structures wherein the electrode or component is a nonferrous like metal.

In one embodiment, the present invention provides a method of stimulating neuro or other biological structures wherein the electrode or component is fixed.

In one embodiment, the present invention provides a method of stimulating neuro or other biological structures wherein the electrode or component is detachable, rotational, or able to be flexed or extended.

In one embodiment, the present invention provides a method of stimulating neural or other biological structures wherein the electrode or component is solidified.

In one embodiment, the present invention provides a method of stimulating neural or other biological structures wherein the electrode or component is in native form.

In one embodiment, the present invention provides a method of stimulating neural or other biological structures wherein the electrode or component is guided is in series.

In one embodiment, the present invention provides a method of stimulating neural or other biological structures wherein the electrode or component is guided to be parallel to each other.

In one embodiment, the present invention provides a method of stimulating neural or other biological structures wherein the electrode or component is guided to be within a matrix to fixed position.

In one embodiment, the present invention provides a method of stimulating neural or other biological structures wherein the electrode or component is guided by magnetism.

In one embodiment, the present invention provides a method of stimulating neural or other biological structures wherein the electrode or component is guided to assemble.

In one embodiment, the present invention provides a method of stimulating neural or other biological structures wherein the electrode or component is guided to detach or separate

In one embodiment, the present invention provides a method of stimulating biological structures, comprising placing one or more electrodes in a fluid or gel like substance or putty like substance; and guiding the placement of the one or more electrodes using a tool with high strength magnet.

In one embodiment, the present invention provides a device of stimulating biological structures, comprising substances carrying one or more micro-chips.

In one embodiment, the present invention provides a method of stimulating neuro or other biological structures having one or more micro-chips forming a matrix in the substance.

In one embodiment, the present invention provides a method of stimulating neuro or other biological structures wherein one or more micro-chips form a matrix in the injected target.

In one embodiment, the present invention provides a method of stimulating neuro or other biological structures wherein non-biological structures, comprising substances carrying one or more micro-chips are used.

In one embodiment, the present invention provides a method of stimulating biological structures, comprising placing micro-chips in a substance, and injecting microchips into biological structures such as one or more nervous systems, one or more organs, the pancreas, one or more cells, blastocyst, or one or more organelles.

Additional objects and advantages of the invention will be set forth in part in the description which follows, and in part will be obvious from the description, or may be learned by practice of the invention. The objects and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

### BRIEF DESCRIPTION OF THE SEVERAL VIEWS OF THE DRAWINGS

In the drawings, which are not necessarily drawn to scale, like numerals may describe substantially similar components throughout the several views. Like numerals having different letter suffixes may represent different instances of substantially similar components. The drawings illustrate generally, by way of example, but not by way of limitation, a detailed description of certain embodiments discussed in the present document.

FIG. 1 illustrates a lower lumbar retrograde placement spinal cord stimulator down the lumbar epidural space and the nerve root and into the plexus for an embodiment of the present invention.

FIG. 2 illustrates a thoracic placement of the spinal cord stimulator for an embodiment of the present invention.

FIG. 3 illustrates the placement of a stimulator along the spinal column and into a peripheral area such as the lumbar nerves for of an embodiment of the present invention.

FIG. 4A illustrates a catheter having multiple stimulators thereon for therapeutic treatment of the upper thoracic area, the DRG area, and peripheral areas for an embodiment of the present invention.

FIG. 4B illustrates an embodiment based on the embodiment shown in FIG. 4A with an additional branch for treatment of a peripheral area.

FIG. 4C illustrates an embodiment of the present invention having a plurality of branches for providing a wide area treatment pattern.

### DETAILED DESCRIPTION OF THE INVENTION

Detailed embodiments of the present invention are disclosed herein; however, it is to be understood that the disclosed embodiments are merely exemplary of the invention, which may be embodied in various forms. Therefore, specific structural and functional details disclosed herein are not to be interpreted as limiting, but merely as a representative basis for teaching one skilled in the art to variously employ the present invention in virtually any appropriately detailed method, structure or system. Further, the terms and phrases used herein are not intended to be limiting, but rather to provide an understandable description of the invention.

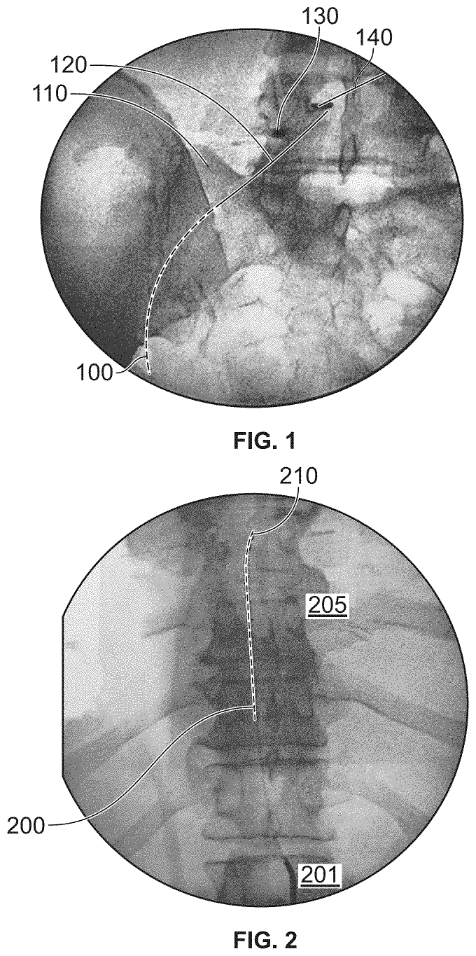

As shown in FIG. 1, in one embodiment, the present invention provides a device and method of treating disorders by placing a stimulator contact device 100 or component into a distal neuro-structure 110 along the nerve root or nerve root branches 120. Contact device 100 may also be applied in the epidural space or blood vessel to reach the desired effect.

In a preferred embodiment, device 100 includes a first stimulator 130 and a second stimulator 140. Stimulator 130 may be aligned to affect the DRG area and stimulator 140 may be positioned to affect another predetermined area.

FIG. 2 illustrates another embodiment of the present invention including device 200 which may be a catheter that is inserted into spinal cord 205. Device 200 may include contact point 201 such as a lead that is inserted into the spinal cord 205. Tip 210 may be positioned distally along the nerve roots. Lead 201 can be located either immediately proximal, distal, or intraparametally position, this can include the stimulation which may or may not affect stimulation of the dorsal root ganglion. The contact can be paced at any point on the nerve through a lead, and such contacts can be achieved through a microchip. The catheter can be internally attached, or individually operated.

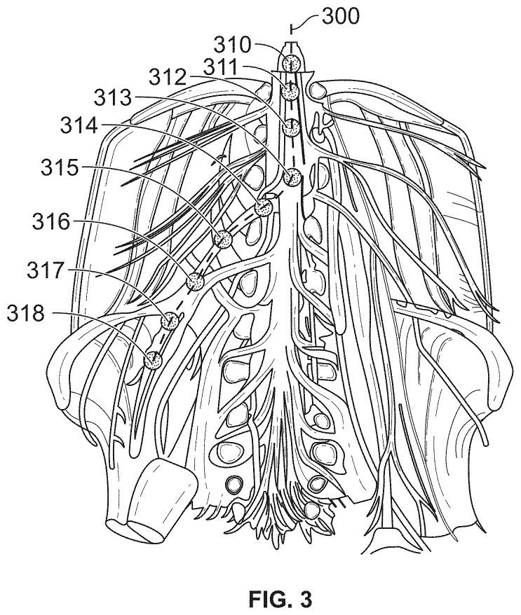

FIG. 3 illustrates the placement of a stimulator along the spinal column and into a peripheral area such as the lumbar nerves for of an embodiment of the present invention. Specifically, device 300, which may be in the form of a catheter, includes a plurality of stimulators 310-318 along its length. In a particular application, this embodiment may be used to treat, through electrical stimulation, portions of the spinal column, such as spinal nerves, by the use of discrete stimulators 310 through 314. In addition, device 300 may include additional discrete stimulators 316-318 that may be used to treat peripheral area such as the lumbar nerves.

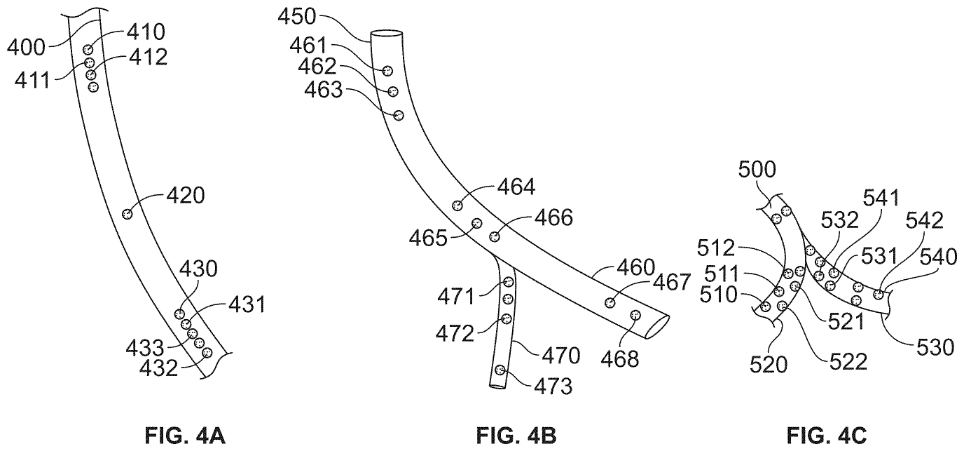

FIGS. 4A-4C illustrate various alternate embodiments of the present invention which provide catheters that may be internally buddle inside a larger, main catheter. The internal catheters can be aligned in the same directions or opposition directions. The internal catheters can branch off from the main catheter and can be detachable from the main catheter. The lead can direct fluid from the main catheter to the smaller internal catheters, or from the smaller catheters, either internal or branched catheter to the larger, main catheter.

In one preferred embodiment, FIG. 4A illustrates a catheter 400 having multiple stimulators thereon for therapeutic treatment of the upper thoracic area by the use of discrete simulators 410-412, the DRG area by the use of discrete simulator 420, and peripheral areas by the use of discrete simulators 430-433 for an embodiment of the present invention. In another preferred embodiment, FIG. 4B illustrates a catheter 450 having a first section of said stimulators 461-463, a second set of distally spaced apart stimulators 464-466 along with another set of spaced apart stimulators 467-468. In addition, main body 460 of catheter 450 includes branch 470 that also includes discrete stimulators 471-473. Branch 470 may be used to treat peripheral areas off of a spinal cord or other major nerve pathways.

Yet another embodiment, as shown in FIG. 4C, the present invention provides catheter 500 that has a plurality of branches 510, 520, 530, and 540. Branch 510 has a plurality of stimulators 511 and 512. Branch 520 has a plurality of stimulators 521 and 522. Branch 530 has a plurality of stimulators 531 and 532. Branch 540 has a plurality of stimulators 541 and 542.

In yet other embodiments, the present invention provides a device and method of treating a disorder of interest, utilizing retrograded approach to place a stimulator contact device or component into a position along the nerve root or branch thereof in a distal neurostructure such that neuro-application inhibits the disorder. This form of neuroaugmentation may be applied in the epidural space proximal to the distal neuro-structure or one or more blood vessels.

The present invention, as discussed above, involves the use of a plurality of discrete stimulators whereby such contact is located either immediately proximal, distal, or in an intraparametally position, you may include the stimulation which may or may not affect stimulation of the dorsal root ganglion. Other areas of stimulation include the foramen which is at the level of T12, or L1, or L2, or L3, or L4, or L5, or any sacral level and it can be unilaterally or bilaterally to affect inhibition the disorder of interest.

In yet other aspects, the present invention relates to compositions, devices, kits and methods for treating neuropathies, Diabetic, or other metabolic or toxic, compressive neuropathies, post viral neuropathies, Post heretic neuropathies, Shingles, myelopathies, cauda equina neuropathies, nerve root neuropathies, sympathetic mediated pain, and related disorders, including inhibiting pain associated with them, or for improving functionality, such as functionality resulting from the loss of motor or sensory function, sympathetic or parasympathetic and/or vagal tone or balance or range or fluidity of motion or muscular function, following or as a result of trauma, neoplasm, cancer, endometriosis, cystitis, Meralgia paresthetica, femoral, or pudendal, or genital, or genitofemoral, or ilioinguinal, or iliohypogastric neuropathy, or from surgery, hernia repair with or without mesh, pelvic, genitourinary, prostate, gynecological, prostatic or groin or other surgery or radiotherapy or small fiber peripheral neuropathy or nerve damage or compromise, or sympathetic dysfunction such as but not limited to at least one of a disorder comprising pain or loss of motor or sensory or other function, sympathetic, parasympathetic or vagal function, balance or tone, or range or fluidity of motion, or muscular function involving one or more areas, of, tissues, spinal cord structures, nerve roots, sympathetic or parasympathetic structures or cauda equina neural structures, or nerve or nerve or ganglion or plexus neural structures or peripheral neural structures in or around the whole or part, superficial or deep, of the thorax, abdomen, pelvis, rectal areas, genitalia, genitourinary system shoulder, back, elbow, wrist, hip, knee, ankle or other joints, or musculature or connective tissue.

Any single one or any combination of these disorders, neuropathies, dysfunctions, conditions or symptoms are treated according to this invention by affecting, augmenting, interrupting, disrupting or otherwise modifying one or more of spinal, nerve root, DRG, ganglial, plexus, sympathetic, parasympathetic or other neural or other anatomic or physiologic related structures or pathways associated with said neuropathy, pain, dysfunction or sympathetic, parasympathetic vagal or other disorder of interest.

The nerve pathway may be inhibited, augmented, blocked or disrupted by any of the following interventions by applying any energy, light, laser, ultrasound, infrasound, radiowave, microwave, induced heat, electrical potential or current, including low level current, or electromagnetic radiation near or to any part or structure of any related neural structure, ganglion, nerve, or nerve pathway. This may be effected externally or internally, via implantable or superficially applied or directed, such as transcutaneous, transmuscular, transcavity, transmucosal or transosteal or periorganal, pericavitary, periliganentary, subcutaneous, submucosal electrical or other neural stimulation, or via an implantable device or portion of device, and preferably a miniaturized electronic stimulation device, induction receiver, or energy generating device, energy receiving or modulation or transmitting device, or stimulator such as a microelectromechanical systems device (MEMS), nanoelectromechanical systems device (NEMS), microchip, magnetic induction device, radio frequency emitting or receiving or modulation device. Use of radiation or visible or non-visible light frequency, x-rays, proton bombardment, ultrasound, infrasound, near infrared or laser, applying heat, applying cold, or mechanical massage; or any other technique for stimulating an organ, tissue or nerve pathway to inhibit the disorder, neuropathy, condition or symptom or any one or more conditions or interventions mentioned herein will be referred to hereinafter as a “Stimulation Technique” and any one or more devices used to stimulate, sense a condition of or monitor a function of an organ, tissue or nerve pathway or for any purpose set forth herein will referred to as a “Stimulation Device”); or stimulation by any suitable means, such as topically, transcutaneously, epidurally with contiguous or noncontiguous, integral or separate transforaminal component to stimulate any portion of the nerve root nerve root, including the DRG, and into a plexus, or even distally to a position in a more peripheral location to effect separate or additive more peripheral or peripheral neurostimulation. A neurostimulation device may be placed near or on the GI tract organs, including the stomach, intestines, liver, pancreas or gall bladder to stimulate secretion of digestive enzymes, influence endocrine function, stimulate pancreatic release of insulin, digestive enzymes, or proliferation of beta cells or enhance their function, alter hepatic lipid, cholesterol, glucose production or modulation, or modulate gastric acid production and the like. A Neurostimulation devices may be thread into or through the epidural space and into and through the neurforamen into any position in the nerve root, near or not near the DRG, or to a plexus or more distal position in a more peripheral branch of the neural structure up to and including a branch or branches of a peripheral nerve, such that any location or locations may be stimulated simultaneously or otherwise with similar or different parameters. Any one or any combination of two or more of these types of interventions will be referred to hereinafter as an “Intervention” or “Interventions”.

Hence, epidural/spinal cord, and peripheral nerve stimulation, neuroaugmentation or neuromodulation involve a distinct nerve or ganglion structure or portion thereof which is targeted by the Interventions. One aspect of this invention targets peripheral nerve fibers or other structures physiologically or anatomically related to a given nerve structure or pathway, with or without having to directly target the specific distinct nerve structure itself. Thus, it is easier, safer and more practical to do and is surprisingly effective. Similarly, spinal, thoracic, splanchnic, sympathetic, parasympathetic or vagal or other stimulation or augmentation can be used to alter physiologic processes including insulin production and resistance, lipid, cholesterol, glucose production and modulation, and beta cell proliferation.

Neurostimulation or related modalities may be used on but not limited to epidural/spinal cord, sympathetic, parasympathetic, vagal, plexus peripheral nerve structures or other neural structure in any location including the cervical, thoracic, lumbar or sacral spinal segments, including foraminal or more distal neural structures, splancnic or related or periorganal neural structures, any part or whole of any plexus or nerve affecting the upper or lower extremities, abdomen, pelvis, GI, or any portion of the spinal cord, sciatic, femoral, tibial sural, perineal, genitofemoral, ilioinguinal, iliohypogastric, lateral femoral cutaneous nerve, lumbar plexus, brachial plexus, or branches thereof, surprascapular nerve, radial nerve, median nerve, peripheral or sympathetic nerves, including branches and small fibers of such nerves, associated with the neuropathy, pain, disorder or dysfunction in any manner to inhibit the neuropathy, pain, disorder or dysfunction.

Peripheral nerve injections, stimulation, neuroaugmentation or neuromodulation involve a distinct nerve or ganglion structure is also targeted by the Interventions. One aspect of this invention targets peripheral nerve fibers or other structures physiologically or anatomically related to a given nerve structure or pathway, with or without having to directly target the specific distinct nerve structure itself. Thus, it is easier, safer and more practical to do and is effective as it was in the example provided.

The epidural space may be accessed in the usual manner and the lead or stimulator steered into the neuroforamen, or it can be done retrograde with the bevel down to access inferior portions, i.e., the sacral segments, or the bevel of the needle may be positioned at the appropriate angle to best direct the lead or device through the neuroforamen.

Another aspect of this invention, with or without stimulation, involves the use of this technique to enhance neural healing, regeneration or synthesis. The damaged, injured or missing segment or segments of the spinal cord, nerve root or peripheral neural, vascular or other structure is identified using MRI, ultrasound, or other imaging, electrophysiologic diagnostics, and or clinical findings. A scaffolding is construct preimplanted with stem cells, Platelet rich plasma, autologous or non-autologous tissue and advanced into the epidural, or subarachnoid space and into, through or adjacent to the damaged portion using imaging, electrophysiologic, or elicited signs. The scaffold will be following the normal course, proximal to distal, if the neuronal, vascular or other structure, and then the scaffolding can entrap by deployment to envelope the damaged area, or a portion can penetrate the peri, or endoneurium or other part of the nerve, or enwrap around and anyeurysmal segment, or penetrate in the endovascular segment of a vascular structure.

Post Herniorraphy Pain is a serious painful condition which has a prevalence of, and which may be debilitating and refractory to treatment. There is a need for more effective options for treating this disorder and other disorders of peripheral nerve that is less traumatic and more easily accomplished than peripheral nerve stimulation techniques and which can offer the benefit a single procedure which can also allow DRG or spinal cord stimulation to be performed using a single trial or implantation procedure.

Herniorrhaphies are performed 800 times a year in the U.S. A male person in the industrialized world faces an up to 27% lifetime risk of requiring surgery for his inguinal hernia. Some 10-12% of these patients were found to report moderate to severe chronic pain after the operation. Chronic postherniorrhapy inguinal pain may be caused by nociceptive, inflammatory or neuropathic changes, the latter depending on whether inguinal nerves are affected. A comparably related pain syndrome may occur after nerve entrapment following a lower abdominal pfannenstiel incision. Both pain entities are also referred to as chronic post-surgical inguinal pain (PSIP) syndromes.

The results of the presently proposed ‘SMASHING’ trial may shed light on a possible viable alternative treatment option once other treatments have failed in therapy resistant patients with PSIP. A very recent consensus protocol does not address this group. Based on a retrospective analysis of 10 CPIP patients who were implanted with DRG stimulation leads, the technique is very promising. Eight of these 10 patients reported >50% pain relief whereas a 77% mean VAS reduction was attained. However, it is obvious that a proper RCT is required using a cross-over design. Firstly, a placebo controlled setting is inappropriate as it is clearly noticeable for patients if a sham device is implanted because of the absence of paresthesia. Secondly, ethics direct that the control group is not to be denied access to the therapy for which a cross-over is offered. Thirdly, a control group is heterogeneous because of the often individually tailored treatments for these therapy-resistant patients. This heterogeneity however mirrors daily clinical practice, and therefore generalization of the forthcoming results will be realistic. Moreover, possible confounding variables are controlled with randomization. The PROCESS study used a similar construction.

It must be appreciated that the validation of various outcomes is of utmost importance in pain treatment studies. Pain reduction scores are subjective whereas the clinical relevance of a 30% or 50% pain reduction is debatable. The presently proposed outcome measurements are linked to improved patient satisfaction, diminished medication usage, daily functioning and sleeping quality and a positive expert's opinion regarding patient's improvement.

One study showed “the prevalence of CP after hernia surgery to be nearly 20%, with significantly increased costs and healthcare resource utilization. While current treatment paradigms are effective for many, there remains a large number of patients that could benefit from an overall approach that includes nonopioid treatments, such as potentially incorporating neurostimulation, for CP that presents post hernia repair.

### Nerves of the Groin

The cutaneous nerves of the lower abdomen and groin that are most frequently implicated in the etiology of persistent groin pain following hernia repair include the ilioinguinal, iliohypogastric, genitofemoral, and lateral femoral cutaneous nerves. These nerves arise from the lumbar plexus and provide cutaneous sensory innervation for the groin, upper hip, and thigh regions.

The ilioinguinal, iliohypogastric, and lateral femoral cutaneous nerves travel along the anterior surface of the quadratus lumborum, and the genitofemoral nerve runs along the anterior surface of the psoas major muscle before piercing the abdominal wall.

The ilioinguinal nerve emerges lateral to the internal ring traveling toward the external ring. The anterior branch of the iliohypogastric nerve is located more medially between the external oblique aponeurosis and the underlying internal oblique muscle.

State of the art approach uses percutaneous entry over the groin, and the stimulation lead is placed too superficially, i.e.. only subcutaneously, and yields less effective stimulation than at a more proximal location along the distal nerve branch, i.e. the entry of the peripheral nerve near its origin point. Further, subcutaneous placement may result in movement if the lead with weight gain, movement, and pressure, and the lead itself may be felt to feel uncomfortable or annoying as its presence can be felt, unlike the technique of the present invention.

Ilioinguinal, Genitofemoral, and Iliohypogastric Nerve Stimulation

Inguinal hernia repair and surgeries in the groin are associated with an 11%-30% incidence of postsurgical chronic pain. 17 Patients undergoing inguinal lymph node resections for melanomas or sarcomas and hernia repairs also may experience ilioinguinal, genitofemoral, and/or iliohypogastric nerve injuries during these procedures. A history and physical evaluation will help define whether the pain is related to ilioinguinal or genitofemoral nerve injuries, or a combination of both. Patients with ilioinguinal nerve injury will complain of burning pain at the upper portion of the scrotum or labia, the superomedial portion of the thigh, and the medial portion of the groin. In contrast, patients with genitofemoral nerve injury will have pain at the bottom of the scrotum and the proximal medial portion of the thigh, and patients with iliohypogastric nerve injury will complain of pain throughout the groin area, extending to the anterior superior spine. These findings can be helpful in determining if a patient will need one or two leads subcutaneously inserted at the level of the anterior superior spine with typical placement above the surgical scar, and just beyond the boundaries of the medial aspect of the scar for patients with genitofemoral nerve injury, or below the scar in those with either iliohypogastric or ilioinguinal nerve injuries.

A middle aged male suffered from post herniorraphy pain and became disabled following hernia repair. He eventually underwent testicle biopsy and surgical neurolysis which worsened his pain. His pain was refractory to treatment, but he noted some improvement of pain following an ilioinguinal nerve block. He underwent spinal cord stimulator trial, and an Infion BSCI lead was advanced to an appropriate position on the ipsilateral thoracic epidural space. This covered part of his pain distribution, and not entirely within that distribution. Therefore, a retrograde approach was used and an Infion lead thread down to the ipsilateral sacral area, but only lower extremity paresthesia not covering his inner thigh, groin, pelvic and abdominal pain were elicited. The lead was withdrawn, and the Touhey needle was rotated and the lead advanced to an area where the DRG was located, stimulation was helpful but not adequately effective in distribution and effect. There were no motor effects anywhere. Next the lead was advanced, with suboptimal stimulation effects note until the lead was very peripheral and visualized in the pelvis. Stimulation was excellent in effect, and in combination with the first lead, the patient had optimum relief. Hence, a combined lead enabling more peripheral transforaminal neurostimulation was felt to be superior to spinal cord stimulation, with or without DRG stimulation, and that a combination of peripheral stimulation with one or both of the aforementioned stimulations is optimal. Further, leads placed in this manner are less uncomfortable as they are not felt by the patient, unlike subcutaneous leads which may feel like a foreign body under a patient's skin.

While the foregoing written description enables one of ordinary skill to make and use what is considered presently to be the best mode thereof, those of ordinary skill will understand and appreciate the existence of variations, combinations, and equivalents of the specific embodiment, method, and examples herein. These include intro as ular, pulmonary, intraviscal, GI, GU, Renal and lymphatic devices, access devices, stenting devices, intracranial and other devices and procedures The disclosure should therefore not be limited by the above described embodiments, methods, and examples, but by all embodiments and methods within the scope and spirit of the disclosure.

What is claimed is:

1. A method of treating a disorder of interest, utilizing retrograded approach to place a stimulator contact device or component to a position along a nerve root or branch thereof or more distal neural structures such that neuro application inhibits the disorder.

2. The method of claim 1, thereby neuroaugmentation is applied in the epidural space proximal to the distal neuro-structure of claim 1.

3. The method of claim 1, thereby neuroaugmentation is applied to a DRG of claim 1.

4. The method of claim 1 wherein one or more contact, device or stimulator component whereby such contact is located either immediately proximal, distal, or intraparametally position, this can include the stimulation which may or may not affect stimulation of the dorsal root ganglion.

5. The method of any of claim 1 wherein the foramen is at the level of T12, or L1, or L2, or L3, or L4, or L5, or any sacral level and it can be unilaterally or bilaterally to affect inhibition the disorder of interest.

6. The method of claim 5, wherein the position of the contact, is frequency, intensity, pulse-width, and/or other parameters, which can be varied among more than one of epidurally placed central contacts, proximal and distal Foraminal contacts DRG level contacts, plexus level contacts or more distally neural loci or topical contacts to maximize efficacy

7. Steerable or advanceable leads or devices to optimize position circumferentially further from anterior motor neural components and/or optimizing parameters to minimize motor stimulation
